(19) United States
(12) Patent Application Publication
(10) Pub. No.: US 2004/0241651 A1
(43) Pub. Date: Dec. 2, 2004

(54) Detection of single nucleotide polymorphisms (snp's) and cytosine-methylations

(76) Inventors: Alexander Olek, Berlin (DE); Christian Piepenbrock, Berlin (DE); Kurt Berlin, Stahnsdorf (DE)

Correspondence Address: KRIEGSMAN & KRIEGSMAN, 665 FRANKLIN STREET, FRAMINGHAM, MA, 01702, US

(21) Appl. No.: 10/257,017

(22) PCT Filed: Apr. 6, 2001
(86) PCT No.: PCT/IB01/00713

(30) Foreign Application Priority Data
Apr. 7, 2000 (DE) 100 19 173.8

(51) Int. Cl. C12Q 1/68; C07H 21/04; C07K 14/00

(52) U.S. Cl. 435/6; 530/350; 536/24.3

(57) ABSTRACT

A set of oligonucleotides or PNA oligomers and a method which is suitable for the detection of cytosine methylations and SNPs in genomic DNA samples is described.

This method serves for the diagnosis and/or prognosis of adverse events for patients or individuals, as well as diseases.

The present invention describes a representative set of oligonucleotides or PNA (peptide nucleic acid) oligomers, which are particularly suitable for the simultaneous detection of SNPs (single nucleotide polymorphisms) and cytosine methylations in genomic DNA samples for distinguishing cell types, as well as a method that is used.

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with high probability with the extent and nature of the methylation of the genes or of the genome. Pathogenic states are also expressed by a modified methylation pattern of individual genes or of the genome.

### Prior Art

The Human Genome Project, the first sequencing of the human genome, will be completed in the next few years. Due to this Project, it will be possible to identify all approximately 100,000 genes. The sequence information opens up unexpected possibilities for the clarification of gene functions. This in turn can become a driving force in pharmacogenetics and pharmacogenomics. Pharmacogenetics and pharmacogenomics relate to the application of medications as a function of a genotype. The effectiveness of medications will be increased in this way. The necessary intermediate step is the determination of polymorphisms and genotypes which are associated with a specific response. Thus, continuously more efficient genotyping methods will be required.

Currently, there are two categories of polymorphic markers, which are utilized for genotyping: microsatellites and single nucleotide polymorphisms (SNPs). Microsatellites are highly polymorphic, i.e., they have a multiple number of alleles. They are characterized in that a repetitive sequence element, with a different number of repetitions for different alleles, is stamped by conserved sequences. On average, there is one microsatellite marker per 1 million bases. A map of 5,000 positioned microsatellite markers was published by CEPH. Microsatellites are genotyped by determining the size of products of a PCR with primers of conserved, flanking sequence. The fluorescently labeled PCR products are separated on gels.

There are comparatively few SNP markers that have been described. A map with 300,000 SNP markers is currently being developed by the SNP Consortium and will be made accessible in the public domain. If SNP markers are identified, they can be assigned genomic positions. It is attempted to map 150,000 SNP markers by the year 2001 (Mashall, E. (1999); Science, 284, 406-407). There is a handful of genotyping methods for SNPs. Several are based on the separation of products on gels, such as the oligonucleotide ligase assay (OLA). The latter is suitable rather for an intermediate throughput. Others rely on pure hybridization, which does not have the same stringency, however. DNA arrays (DNA chips) are suitable for the analysis of a large number of SNPs in a limited number of individuals. Up to now, examples have been shown, in which 1,500 SNPs were genotyped on one DNA chip. The true strength of DNA chips lies in approaches, such as resequencing und expression analysis. Approaches which apply primer extension have been shown (Head, S. R. et al., (1999); Mol Cell Probes, 13(2), 81-87). If one is working with fluorescently labeled terminator bases, these chips have the advantage that the results can be compiled with a simple ELISA reading device.

There are several SNP genotyping methods, which use mass spectrometry for analysis. These have the basic advantage that the allele-specific products are a physical representation of the product and not, e.g., a fluorescent signal that is indirectly assigned to the product. Matrix-assisted laser desorption/ionization time-of flight mass spectrometry (MALDI) has revolutionized the analysis of biomolecules (Karas, M. & Hillenkamp, F., Anal. Chem. 60, 2299-2301 (1988)). MALDI has been applied in different variants to the analysis of DNA. The variants extend from primer extension to sequencing (Liu, Y. -H., et al. Rapid Commun. Mass Spectrom. 9, 735-743 (1995); Ch'ang, L. -Y., et al. Rapid Commun. Mass Spectrom. 9, 772-774 (1995); Little, D. P., et al. J. Mol. Med. 75, 745-750 (1997); Haff, L. & Smirnov, I. P. Genome Res. 7, 378-388 (1997), Fei, Z., Ono, T. & Smith, L. M. Nucleic Acids Res. 26, 2827-2828 (1998); Ross, P., Hall, L., Smirnov, I. & Haff, L. Nature Biotech. 16, 1347-1351 (1998); Ross, P. L., Lee, K. & Beigrader, P. Anal. Chem. 69, 4197-4202 (1997); Griffin, T. J., Tang, W. & Smith, L. M. Nature Biotech. 15, 1368-1372 (1997)). The greatest disadvantage of all these methods is that they all require a basic purification of the products prior to the MALDI analysis. Spin column purification or the use of magnetic bead technology or reversed-phase purification is necessary.

The analysis of DNA in MALDI is very dependent on the charge state of the product. A 100-fold improvement of sensitivity in the MALDI analysis can be achieved by the fact that the charge state is controlled on the product to be analyzed, so that only a slight positive or negative excess charge is present. The products modified in this way are also essentially less susceptible to the formation of adducts (e.g. with Na and K, Gut, I. G. and Beck, S. (1995) Nucleic Acids Res., 23, 1367-1373; Gut, I. G., Jeffery, W. A., Pappin, D. J. C. and Beck, S. Rapid Commun. Mass Spectrom., 11, 43-50 (1997)). An SNP genotyping method, which makes use of these conditions, with the name “GOOD Assay” has been proposed recently (Sauer, S. et al., Nucleic Acids Research, Methods online, 2000, 28, el 3).

The most frequent covalently modified base in the DNA of eukaryotic cells is 5-methylcytosine. For example, it plays a role in the regulation of transcription, genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines, is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which will now be fully utilized. The prior art, which concerns sensitivity, is defined by a method that incorporates the DNA to be investigated in an agarose matrix, so that the diffusion and renaturation of the DNA is prevented (bisulfite reacts only on single-stranded DNA) and all precipitation and purification steps are replaced by rapid dialysis (Olek, A. et al., Nucl. Acids Res. 1996, 24, 5064-5066). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix. An overview of other known possibilities for detecting 5-methylcytosines can be derived from the following review article: Rein, T., DePamphilis, M. L., Zorbas, H., Nucleic Acids Res. 1998, 26, 2255. With just a few exceptions (e.g. Zechnigk, M. et al., Eur. J. Hum. Gen. 1997, 5, 94-98), the bisulfite technique has only been applied in research. However, short, specific segments of a known gene are always amplified after a bisulfite treatment and either completely sequenced (Olek, A. und Walter, J., Nat. Genet. 1997, 17, 275-276) or individual cytosine positions are detected by a “primer extension reaction” (Gonzalgo, M. L. and Jones, P. A., Nucl. Acids Res. 1997, 25, 2529-2531, WO Patent 95/00669) or an enzyme step (Xiong, Z. and Laird, P. W., Nucl. Acids. Res. 1997, 25, 2532-2534). Detection by hybridization has also been described (Olek et al., WO-A 99/28498).

Other publications which are concerned with the application of the bisulfite technique for the detection of methylation in the case of individual genes are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioassays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261, WO-A 97/46705 and WO-A 95/15373.

A review of the prior art in oligomer array production can be derived from a special edition of Nature Genetics that appeared in January 1999 (Nature Genetics Supplement, Volume 21, January 1999) and the literature cited therein.

Probes with multiple fluorescent labels are used for scanning an immobilized DNA array. Particularly suitable for fluorescent labeling is the simple introduction of Cy3 und Cy5 dyes at the 5′-OH of the respective probe. The fluorescence of the hybridized probes is detected, for example, by means of a confocal microscope. The dyes Cy3 and Cy5, in addition to many others, can be obtained commercially.

### Statement of the Problem

The present invention will offer a set of oligonucleotides or PNA oligomers and a method which are suitable for the simultaneous detection of SNPs (single nucleotide polymorphisms) and cytosine methylations in genomic DNA samples.

### DESCRIPTION

The problem is thus solved by a set of oligonucleotides or PNA (peptide nucleic acid) oligomers for the detection of single nucleotide polymorphisms (SNPs, single nucleotide polymorphisms) and for the detection of the state of cytosine methylation in chemically pretreated genomic DNA, wherein the base sequences are selected from SEQ-ID: 1 to SEQ-ID: 382046.

It is further provided according to the invention that the set according to the invention contains both base sequences with the SEQ-ID: 1 to SEQ ID: 382046 itself and/or contains the named sequences with SEQ-ID: 1 to SEQ-ID: 382046 by extension, truncation or modification. The set according to the invention may thus be comprised of unmodified sequences according to the invention and/or sequences modified in the way according to the invention.

The present invention describes a set of oligomer probes (oligonucleotides and/or PNA oligomers) for the detection of single nucleotide polymorphisms and/or the state of cytosine methylation in chemically pretreated genomic DNA, which most preferably comprises at least 10 of the listed oligonucleotides or PNA sequences selected from the sequences SEQ-ID: 1 to SEQ-ID: 382046, or at least 10 PNA oligomers or oligonucleotide sequences, which in turn contain the sequences listed therein, namely the sequences SEQ-ID: 1 to SEQ-ID 382046.

In another variant of the method, the set of oligomer probes (oligonucleotides and/or PNA oligomers) for the detection of single nucleotide polymorphisms and/or the state of cytosine methylation in chemically pretreated genomic DNA comprises at least 100 oligonucleotides or PNA sequences selected from the sequences SEQ-ID: 1 to SEQ-ID: 382046, or, however at least 100 PNA oligomers or oligonucleotide sequences, which in turn contains the sequences listed therein, namely the sequences SEQ-ID: 1 to SEQ-ID: 382046.

Particularly preferred, the set of oligonucleotides for the detection of single nucleotide polymorphisms and the state of cytosine methylation in the chemically pretreated genomic DNA is characterized in that the base sequences mostly at the 5′ end and/or at the 3′ end can each be extended by another base, whereby the bases can be A, T or C.

Particularly preferred, the set of oligonucleotides for the detection of single nucleotide polymorphisms and the state of cytosine methylation in the chemically pretreated genomic DNA in turn is characterized in that the base sequences mostly at the 5′ end and/or at the 3′ end can each be extended by another base, whereby the bases can be A, T or G.

The set of oligonucleotides for the detection of single nucleotide polymorphisms and the state of cytosine methylation in the chemically pretreated genomic DNA is preferably characterized in that the base sequences mostly at the 5′ end and/or at the 3′ end can each be extended by at least two other bases, whereby the bases can be A, T or C.

The set of oligonucleotides for the detection of single nucleotide polymorphisms and the state of cytosine methylation in the chemically pretreated genomic DNA is preferably characterized in that the base sequences mostly at the 5′ end and/or at the 3′ end can each be extended by at least two other bases, whereby the bases can be A, T or G.

That the set of PNA (peptide nucleic acid) oligomers for the detection of single nucleotide polymorphisms and the state of cytosine methylation in the chemically pretreated genomic DNA is most preferably characterized in that a nucleobase is omitted at the 5′ end and/or at the 3′ end of the oligomer.

The set of PNA (peptide nucleic acid) oligomers for the detection of single nucleotide polymorphisms and the state of cytosine methylation in the chemically pretreated genomic DNA is preferably characterized in that at least two nucleobases are omitted each time at the 5′ end and/or at the 3′ end of the oligomer.

A representative set of oligonucleotides and/or PNA oligomers, comprising oligomers and/or oligonucleotides according to the sequences SEQ-ID: 1 to SEQ-ID: 382046, will be used for the detection of cytosine methylations and single nucleotide polymorphisms in genomic DNA for distinguishing cell types or tissues or for the investigation of cell differentiation. For this purpose, the following process steps are sequentially conducted:

In the first step of the method, a genomic DNA sample is chemically treated in such a way that cytosine bases unmethylated at the 5′ position are converted to uracil, thymine or another base dissimilar to cytosine in its hybridizing behavior.

The genomic DNA to be analyzed is preferably obtained from the usual sources for DNA, such as, e.g., cell lines, blood, sputum, stool, urine, cerebrospinal fluid, tissue embedded in paraffin, histological slides and all possible combinations thereof.

Preferably, the above-described treatment of genomic DNA is conducted with bisulfite (hydrogen sulfite, disulfite) and subsequent alkaline hydrolysis for this purpose, which leads to a conversion of unmethylated cytosine nucleobases to uracil.

In a preferred variant of the method, the amplification is conducted by means of the polymerase chain reaction (PCR), whereby a heat-stable DNA polymerase is used.

In a second method step, more than ten different fragments, each of which is less than 2000 base pairs long, are amplified [simultaneously] from the chemically pretreated genomic DNA with the use of synthetic oligonucleotides as primers.

In a particularly preferred variant of the method, the oligonucleotides or PNA oligomers are bound to defined sites on a solid phase.

In another preferred variant of the method, different oligonucleotides and/or PNA oligomer sequences are arranged on a planar solid phase in the form of a rectangular or hexagonal grid.

The solid-phase support surface is preferably comprised of silicon, glass, polystyrene, aluminum, steel, iron, copper, nickel, silver, or gold.

In the third step of the method, the amplified products are hybridized to a set of oligonucleotides or PNA oligomers, which comprise at least 10 of the above-named sequences, namely the sequences of SEQ-ID: 1 to SEQ-ID: 382046.

In a preferred variant of the method, the amplification of several DNA segments is conducted in one reaction vessel.

In a preferred variant of the method, the base sequences of the set of oligonucleotides according to the invention, namely the sequences SEQ-ID: 1 to SEQ-ID: 382046 are each extended mostly at the 5′ end and/or at the 3′ end by one additional base, wherein the bases can be either A, T or G.

In a preferred variant of the method, the base sequences of the set of oligonucleotides according to the invention are each extended mostly at the 5′ end and/or at the 3′ end by at least two additional bases, wherein the bases can be either A, T or C.

In a preferred variant of the method, the base sequences of the set of oligonucleotides according to the invention for the detection of single nucleotide polymorphisms and of the state of cytosine methylation are each extended mostly at the 5′ end and/or at the 3′ end by at least two additional bases, wherein the bases can be either A, T or G.

In a preferred variant of the method, a set of PNA oligomers comprised of the above-named base sequences is used, wherein one nucleobase is omitted each time at the 5′ end and/or at the 3′ end of the oligomer.

In a preferred variant of the method, a set of PNA oligomers comprised of the above-named base sequences is used, wherein at least two nucleobases are omitted each time at the 5′ end and/or the 3′ end of the oligomer.

In another preferred variant of the method, at least 10 of the above-named oligonucleotides or PNA sequences, namely selected from the sequences SEQ ID: 1 to SEQ-ID: 382046, are used for the detection of the state of cytosine methylation, or, however, at least 10 PNA oligomer or oligonucleotide sequences, which in turn contain the above-named sequences, namely the sequences SEQ-ID: 1 to SEQ-ID: 382046, [are used].

In another preferred variant of the method, at least 100 of the above-named oligonucleotides or PNA sequences, namely selected from the sequences SEQ-ID: 1 to SEQ-ID: 382046, are used for the detection of the state of cytosine methylation, or, however, at least 100 PNA oligomer or oligonucleotide sequences, which in turn contain the above-named sequences, namely the sequences SEQ-ID: 1 to SEQ-ID: 382046, [are used].

In another preferred variant of the method, at least one primer is bound to a solid phase.

In still another preferred variant of the method, different amplified products are arranged on the solid phase in the form of a rectangular or hexagonal grid.

This solid-phase support surface is preferably comprised of silicon, glass, polystyrene, aluminum, steel, iron, copper, nickel, silver, or gold.

In the last step of the method, the hybridized amplified products are detected. The labelings introduced on the amplified products can be identified at any position of the solid phase on which an oligonucleotide sequence is found.

In a preferred variant of the method, the labels of the amplified products are fluorescent labels.

In a preferred variant of the method, the labels of the amplified products are radionuclides.

In another preferred variant of the method, the amplified products bear removable mass labels, which are detected in a mass spectrometer.

In another preferred variant of the method, the amplified products, fragments of the amplified products or probes complementary to the amplified products are detected in the mass spectrometer.

In another preferred variant of the method, the fragments produced have a single positive or negative net charge in the mass spectrometer for better detectability.

The set of oligonucleotides and/or PNA oligomers according to the invention preferably is used for the diagnosis and/or prognosis of adverse events for patients or individuals. Preferably, the set of oligonucleotides and/or PNA oligomers according to the invention serves for the diagnosis and/or prognosis of adverse events for patients or individuals, whereby these adverse events belong to at least one of the following categories:

undesired drug interactions; cancer diseases; CNS malfunctions, damage or disease; symptoms of aggression or behavioral disturbances; clinical, psychological and social consequences of brain lesions; psychotic disturbances and personality disorders; dementia and/or associated syndromes;

cardiovascular disorder, malfunction and damage; malfunction, damage or disorder of the gastrointestinal tract; malfunction, damage or disorder of the respiratory system; lesion, inflammation, infection, immunity and/or convalescence; malfunction, damage or disease of the body as an abnormality in the development process; malfunction, damage.or disorder of the skin, the muscles, the connective tissue or the bones; endocrine and metabolic malfunction, damage or disorder; headaches or sexual malfunctions.

Preferably, the set of oligonucleotides according to the invention and/or PNA oligomers is used for distinguishing cell types or tissues or for investigating cell differentiation.

Another subject of the invention is a kit, which contains at least 10 oligonucleotides or PNA oligomers and primers for the production of amplified products as well as instructions for conducting the method.

According to the invention, a set of oligonucleotides is preferred for the detection of single nucleotide polymorphisms (SNPs, single nucleotide polymorphisms) and of the state of cytosine methylation in chemically pretreated genomic DNA, wherein the base sequences mostly at the 5′ end and/or at the 3′ end can each be extended by another base, whereby the bases can be either A, T or C.

According to the invention, a set of oligonucleotides is preferred for the detection of single nucleotide polymorphisms (SNPs, single nucleotide polymorphisms) and of the state of cytosine methylation in chemically pretreated genomic DNA, wherein the base sequences mostly at the 5′ end and/or at the 3′ end can each be extended by another base, whereby the bases can be either A, T or G.

According to the invention, a set of oligonucleotides is preferred for the detection of single nucleotide polymorphisms (SNPs, single nucleotide polymorphisms) and of the state of cytosine methylation in chemically pretreated genomic DNA, wherein the base sequences mostly at the 5′ end and/or at the 3′ end can each be extended by at least two additional bases, wherein the bases can be either A, T or C.

According to the invention, a set of oligonucleotides is preferred for the detection of single nucleotide polymorphisms (SNPs, single nucleotide polymorphisms) and of the state of cytosine methylation in chemically pretreated genomic DNA, wherein the base sequences mostly at the 5′ end and/or at the 3′ end can each be extended by at least two additional bases, wherein the bases can be either A, T or G.

According to the invention, a set of PNA (peptide nucleic acid) oligomers is preferred for the detection of single nucleotide polymorphisms (SNPs, single nucleotide polymorphisms) and of the state of cytosine methylation in chemically pretreated genomic DNA, wherein each time a nucleobase is omitted at the 5′ end and/or at the 3′ end of the oligomer.

According to the invention, a set of PNA (peptide nucleic acid) oligomers is preferred for the detection of single nucleotide polymorphisms (SNPs, single nucleotide polymorphisms) and of the state of cytosine methylation in chemically pretreated genomic DNA, wherein each time at least two nucleobases are omitted at the 5′ end and/or at the 3′ end of the oligomer.

A set of oligomer probes (oligonucleotides and/or PNA oligomers) is preferred according to the invention for the detection of the cytosine methylation state and/or of single nucleotide polymorphisms in chemically pretreated genomic DNA, comprising at least 10 of the above-named oligonucleotide or PNA sequences.

A set of oligomer probes (oligonucleotides and/or PNA oligomers) is preferred according to the invention for the detection of the cytosine methylation state and/or of single nucleotide polymorphisms in chemically pretreated genomic DNA, comprising at least 100 of the above-named oligonucleotide or PNA sequences.

The subject of the present invention is also a method for the analysis of a representative set of cytosine methylations and single nucleotide polymorphisms in genomic DNA samples for distinguishing of cell types.

In the first step of the method, unmethylated cytosine bases at the 5-position in a genomic DNA sample are converted by chemical treatment to uracil, thymidine or another base that is dissimilar to cytosine in its hybridization behavior.

In the second step of the method, more than ten different fragments, each of which is less than 2000 base pairs long, are amplified [simultaneously] from this chemically treated genomic DNA with the use of synthetic oligonucleotides as primers.

In the third step of the method, the amplified products are hybridized to a set of oligonucleotides or PNA oligomers comprising at least 10 of the above-named sequences selected from the sequences SEQ-ID: 1 to SEQ ID: 382046, or, however, sequences which have been extended, truncated, or modified in the above-described way.

In the fourth step of the method, the non-hybridized amplified products are removed.

In the last step of the method, the hybridized amplified products are detected.

It is preferred according to the invention that the chemical treatment is conducted by means of a solution of a bisulfite, hydrogen sulfite or disulfite.

It is preferred according to the invention that the amplification is conducted by means of a polymerase chain reaction (PCR).

It is preferred according to the invention that the oligonucleotides or PNA oligomers are bound to defined sites on a solid phase.

It is preferred according to the invention that different oligonucleotides and/or PNA oligomer sequences are arranged on a planar solid phase in the form of a rectangular or hexagonal grid.

It is preferred according to the invention that the labelings introduced on the amplified products can be identified at any position of the solid phase on which an oligonucleotide sequence is found.

It is preferred according to the invention that at least one primer is bound to a solid phase in the amplification.

It is preferred according to the invention that different amplified products are arranged on the solid phase in the form of a rectangular or hexagonal grid.

It is preferred according to the invention that the labels of the amplified products are fluorescent labels.

It is preferred according to the invention that the labels of the amplified products are radionuclides.

It is preferred according to the invention that the amplified products bear removable mass labels, which are detected in a mass spectrometer.

It is preferred according to the invention that the amplified products, fragments of the amplified products or probes complementary to the amplified products are detected in the mass spectrometer.

It is preferred according to the invention that for improved detectability in the mass spectrometer, the produced fragments have a single positive or negative net charge.

It is preferred according to the invention that detection is conducted and visualized by means of matrix-assisted laser desorption/ionization mass spectrometry (MALDI) or by means of electrospray mass spectrometry (ESI).

It is preferred according to the invention that the polymerases are heat-stable DNA polymerases.

It is preferred according to the invention that the amplification of several DNA segments is conducted in one reaction vessel.

It is preferred according to the invention that the solid-phase support surface is comprised of silicon, glass, polystyrene, aluminum, steel, iron, copper, nickel, silver, or gold.

It is preferred according to the invention that the genomic DNA has been obtained from a DNA sample, wherein sources for DNA include, e.g., cell lines, blood, sputum, stool, urine, cerebrospinal fluid, tissue embedded in paraffin, histological slides and all possible combinations thereof.

The subject of the invention is also the use of a set of at least 10 of the above-named oligonucleotides and/or PNA oligomers, selected from the sequences SEQ-ID: 1 to SEQ-ID: 382046, or, however, of at least 10 oligomers or oligonucleotides which contain the above-named sequences, for the diagnosis and/or prognosis of adverse events for patients or individuals.

The use of a set of at least 10 of the above-named oligonucleotides and/or PNA oligomers, selected from the sequences SEQ-ID: 1 to SEQ-ID: 382046, or, however, of at least 10 oligomers or oligonucleotides which contain the above-named sequences, is preferred according to the invention for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain lesions; psychotic disturbances and personality disorders; dementia and/or associated syndromes; cardiovascular disorder, malfunction and damage; malfunction, damage or disorder of the gastrointestinal tract; malfunction, damage or disorder of the respiratory system; lesion, inflammation, infection, immunity and/or convalescence; malfunction, damage or disease of the body as an abnormality in the development process; malfunction, damage or disorder of the skin, the muscles, the connective tissue or the bones; endocrine and metabolic malfunction, damage or disorder; headaches or sexual malfunctions.

The use of a set of at least 10 of the above-named oligonucleotides and/or PNA oligomers, selected from the sequences SEQ-ID: 1 to SEQ-ID: 382046, or, however, of at least 10 oligomers or oligonucleotides which contain the above-named sequences, is preferred according to the invention for the distinguishing of cell types or tissues or for the investigation of cell differentiation.

The subject of the present invention is also a kit containing at least 10 of the above-named oligonucleotides and/or PNA oligomers, selected from the sequences SEQ-ID: 1 to SEQ-ID: 382046, or, however, of at least 10 oligomers or oligonucleotides which contain the above-named sequences, and primers for producing the amplified products as well as instructions for conducting the method according to the invention.

The sequence protocol containing the sequences SEQ-ID: I to SEQ ID: 382046 is attached to the International Application in electronically readable form and is a component of this Application.

1. A set of oligonucleotides or PNA (peptide nucleic acid) oligomers for the detection of single nucleotide polymorphisms (SNPs, single nucleotide polymorphisms) and for the detection of the cytosine methylation state in chemically pretreated genomic DNA, selected from the base sequences SEQ-ID: 1 to SEQ-ID: 382046.

2. The set of oligonucleotides for the detection of single nucleotide polymorphisms (SNPs, single nucleotide polymorphisms) and of the state of cytosine methylation in chemically pretreated genomic DNA according to claim 1, further characterized in that the base sequences mostly at the 5′ end and/or at the 3′ end can each be extended by another base, whereby the bases can be either A, T or C.

3. The set of oligonucleotides for the detection of single nucleotide polymorphisms (SNPs, single nucleotide polymorphisms) and of the state of cytosine methylation in chemically pretreated genomic DNA according to claim 1, further characterized in that the base sequences mostly at the 5′ end and/or at the 3′ end can each be extended by another base, whereby the bases can be either A, T or G.

4. The set of oligonucleotides for the detection of single nucleotide polymorphisms (SNPs, single nucleotide polymorphisms) and of the state of cytosine methylation in chemically pretreated genomic DNA according to claim 1, further characterized in that the base sequences mostly at the 5′ end and/or at the 3′ end can each be extended by at least two additional bases, whereby the bases can be either A, T or C.

5. The set of oligonucleotides for the detection of single nucleotide polymorphisms (SNPs, single nucleotide polymorphisms) and of the state of cytosine methylation in chemically pretreated genomic DNA according to claim 1, further characterized in that the base sequences mostly at the 5′ end and/or at the 3′ end can each be extended by at least two additional bases, whereby the bases can be either A, T or G.

6. The set of PNA (peptide nucleic acid) oligomers for the detection of single nucleotide polymorphisms (SNPs, single nucleotide polymorphisms) and of the state of cytosine methylation in chemically pretreated genomic DNA according to claim 1, further characterized in that a nucleobase is omitted each time at the 5′ end and/or at the 3′ end of the oligomer.

7. The set of PNA (peptide nucleic acid) oligomers for the detection of single nucleotide polymorphisms (SNPs, single nucleotide polymorphisms) and of the state of cytosine methylation in chemically pretreated genomic DNA according to claim 1, further characterized in that at least two nucleobases are omitted each time at the 5′ end and/or at the 3′ end of the oligomer.

8. The set of oligomer probes (oligonucleotides and/or PNA oligomers) for the detection of the state of cytosine methylation and/or of single nucleotide polymorphisms in chemically pretreated genomic DNA, comprising at least 10 of the oligonucleotide or PNA sequences of claim 1.

9. The set of oligomer probes (oligonucleotides and/or PNA oligomers) for the detection of the state of cytosine methylation and/or of single nucleotide polymorphisms in chemically pretreated genomic DNA, comprising at least 100 of the oligonucleotide or PNA sequences of claim 1.

10. A method for the analysis of a representative set of cytosine methylations and single nucleotide polymorphisms in genomic DNA samples for the distinguishing of cell types, hereby characterized in that the following steps are conducted:
a) unmethylated cytosine bases at the 5-position in a genomic DNA sample are converted by chemical treatment to uracil, thymidine or another base that is dissimilar to cytosine in its hybridization behavior;
b) more than ten different fragments, each of which is less than 2000 base pairs long, are amplified simultaneously from this chemically treated genomic DNA with the use of synthetic oligonucleotides as primers;
c) the amplified products are hybridized to a set of oligonucleotides or PNA oligomers, comprising at least 10 sequences of claim 1;
d) the non-hybridized amplified products are removed;
e) the hybridized amplified products are detected.

11. The method according to claim 10, further characterized in that the chemical treatment is conducted by means of a solution of a bisulfite, hydrogen sulfite or disulfite.

12. The method according to claim 10, further characterized in that the amplification is conducted by means of the polymerase chain reaction (PCR).

13. The method according to claim 10, further characterized in that the oligonucleotides or PNA oligomers are bound to defined sites on a solid phase.

14. The method according to claim 13, further characterized in that different oligonucleotide and/or PNA oligomer sequences are arranged on a planar solid phase in the form of a rectangular or hexagonal grid.

15. The method according to claim 13, further characterized in that the labelings introduced on the amplified products can be identified at any position of the solid phase on which an oligonucleotide sequence is found.

16. The method according to claim 10, further characterized in that at least one primer is bound to a solid phase in the amplification.

17. The method according to claim 16, further characterized in that different amplified products are arranged on the solid phase in the form of a rectangular or hexagonal grid.

18. The method according to claim 10, further characterized in that the labels of the amplified products are fluorescent labels.

19. The method according to claim 10, further characterized in that the labels of the amplified products are radionuclides.

20. The method according to claim 10, further characterized in that the amplified products bear detachable mass labels, which are detected in a mass spectrometer.

21. The method according to claim 10, further characterized in that the amplified products, fragments of the amplified products, or probes complementary to the amplified probes are detected in the mass spectrometer.

22. The method according to claim 20, further characterized in that, for better detectability in the mass spectrometer, the produced fragments have a single positive or negative net charge.

23. The method according to claim 20, further characterized in that the detection is conducted and visualized by means of matrix-assisted laser desorption/ionization mass spectrometry (MALDI) or by means of electrospray mass spectrometry (ESI).

24. The method according to claim 10, wherein the polymerases are heat-stable DNA polymerases.

25. The method according to claim 10, further characterized in that the amplification of several DNA segments is conducted simultaneously in one reaction vessel.

26. The method according to claim 14, further characterized in that the solid-phase support surface is comprised of silicon, glass, polystyrene, aluminum, steel, iron, copper, nickel, silver, or gold.

27. The method according to claim 10, wherein the genomic DNA has been obtained from a DNA sample, wherein sources for DNA include, e.g., cell lines, blood, sputum, stool, urine, cerebrospinal fluid, tissue embedded in paraffin, histological slides and all possible combinations thereof.

28. Use of a set of oligonucleotides and/or PNA oligomers according to claim 1 for the diagnosis and/or prognosis of adverse events for patients or individuals.

29. The use of a set of oligonucleotides and/or PNA oligomers according to claim 28 for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain lesions; psychotic disturbances and personality disorders; dementia and/or associated syndromes; cardiovascular disorder, malfunction and damage; malfunction, damage or disorder of the gastrointestinal tract; malfunction, damage or disorder of the respiratory system; lesion, inflammation, infection, immunity and/or convalescence; malfunction, damage or disease of the body as an abnormality in the development process; malfunction, damage or disorder of the skin, the muscles, the connective tissue or the bones; endocrine and metabolic malfunction, damage or disorder; headaches or sexual malfunctions.

30. The use of a set of oligonucleotides and/or PNA oligomers according to claim 1 for the investigation of cell types or tissues or for the investigation of cell differentiation.

31. A kit containing at least 10 oligonucleotides or PNA oligomers for the detection of single nucleotide polymorphisms (SNPs, single nucleotide polymorphisms) and for the detection of the cytosine methylation state in chemically pretreated genomic DNA, selected from the base sequences SEQ-ID: 1 to SEQ-ID: 382046, primers for producing the amplified products and instructions for conducting the method according to one of claims 10 to 27.
